(19) United States
(12) Patent Application Publication
(10) Pub. No.: US 2004/0105848 A1
(43) Pub. Date: Jun. 3, 2004

(54) Enteric dialysis compositions and methods

(76) Inventor: Natarajan Ranganathan, Broomall, PA (US)

Correspondence Address: Licata & Tyrrell P.C., 66 E. Main Street, Marlton, NJ, 08053, US

(21) Appl. No.: 10/676,558

(22) Filed: Sep. 30, 2003

Related U.S. Application Data
(63) Continuation-in-part of application No. 09/855,346, filed on May 15, 2001, now Pat. No. 6,706,287.
Application No. 09/855,346 is a continuation-in-part of application No. 09/557,011, filed on Apr. 20, 2000, now Pat. No. 6,706,263.
(60) Provisional application No. 60/131,774, filed on Apr. 30, 1999.

(51) Int. Cl. A61K 45/00

(52) U.S. Cl. 424/93.44

(57) ABSTRACT

The present invention provides compositions and methods of using these compositions for enteric dialysis to treat renal, hepatic and gastrointestinal diseases by eliminating toxins and other metabolic waste products and reducing or retarding undesirable bacterial over growth. The compositions of the present invention comprise a probiotic. These compositions are useful in treating renal and hepatic diseases and bacterial overgrowth in the gastrointestinal tract.

### CROSS REFERENCE TO RELATED APPLICATIONS

This application is a continuation-in-part of U.S. patent application Ser. No. 09/855,346 filed May 15, 2001 which is a continuation-in-part of U.S. patent application Ser. No. 09/557,011 filed Apr. 20, 2000, which is a conversion of U.S. Provisional Application 60/131,774 filed Apr. 30, 1999.

### INTRODUCTION

This work is partly funded from the Small Business Innovation Research (SBIR) grant award from the National Institutes of Diabetes, Digestive and Kidney (NIDDK) Diseases, National Institute of Health (NIH), Bethesda, Md. Grant No: 1R44DK61873-01.

### FIELD OF THE INVENTION

The invention relates to compositions and methods of using these compositions for enteric dialysis to treat renal, hepatic and gastrointestinal diseases by eliminating toxins and other metabolic waste products and reducing or retarding undesirable bacterial over growth. The compositions of the present invention comprise a probiotic. These compositions are useful in treating renal and hepatic diseases and bacterial overgrowth in the gastrointestinal tract.

### BACKGROUND OF THE INVENTION

Kidney disease is ranked fourth among the major diseases in the United States afflicting over 20 million Americans. More than 90,000 patients die each year because of kidney diseases. In recent years the number of chronic kidney failure patients has increased about 11 percent annually. About 80,000 Americans on dialysis die of various complications each year and more than 27,000 are on waiting lists for kidney transplants each year with only about 11,000 of these patients receiving transplants. Further, nearly 350,000 Americans suffer from end stage renal disease (ESRD), which is the final stage in chronic renal failure.

In normal, healthy humans, metabolic waste nitrogen is primarily excreted via the kidneys such as urea, uric acid, creatinine etc. in the urine. However, in individuals with kidney disease, as well as a number of other diseases such as inborn errors in urea cycle enzyme deficit, waste nitrogen accumulates in the body thereby manifesting toxic symptoms. Hyperammonium can lead to mental retardation and, in severe cases, coma.

Currently hemo- or peritoneal-dialysis and renal transplant are the only treatment modalities. However, the economic costs of these treatment modalities are extremely high. For example, in 1996 in the United States alone, the annual cost of ESRD treatment was over 14 billion dollars. In developing and underdeveloped countries with low health care budgets, ESRD patients are deprived access to such treatments due to their high costs. Accordingly, there is a need for alternative modalities of treatment for uremia.

Kidney disfunction often a consequence of other multi-system disorders such as diabetes or artherosclerosis has a broad prevalence afflicting as many as twenty million Americans. Prior to progression to end stage renal disease (ESRD) patients in “affluent” nations are treated with a kidney transplant or dialysis most commonly hemodialysis performed thrice weekly. Unfortunately, the high expense of hemodialysis is unaffordable by the majority of those afflicted with ESRD.

A number of treatment attempts have been based on the use of the bowel as a substitute for kidney function. During a normal digestive process the gastrointestinal tract delivers nutrients and water to the bloodstream and eliminates some waste products and undigested materials through the bowel. The intestinal wall regulates absorption of nutrients, electrolytes, water and certain digestive aiding substances such as bile acids. The intestinal wall also acts as a semi-permeable membrane allowing small molecules to pass from the intestinal tract into the bloodstream and preventing larger molecules from entering the circulation.

Nitrogenous wastes such as urea, creatinine and uric acid, along with several other small and medium molecular weight compounds, flow into the small intestine and equilibrate across the small intestine epithelium. Studies of intestinal dialysis have shown a daily flow of 71 grams of urea, 2.9 grams of creatinine, 2.5 grams of uric acid and 2.0 grams of phosphate into the intestinal fluid (Sparks, R. E. Kidney Int. Suppl. 1975 Suppl 3, 7:373-376). Accordingly, various invasive and noninvasive attempts including external gut fistula, intestinal dialysis, induced diarrhea, and administration of oral sorbents and/or encapsulated urease enzyme have been made to extract uremic waste from the gastrointestinal tract (Twiss, E. E. and Kolff, W. J. JAMA 1951 146:1019-1022; Clark et al. Trans. Am. Soc. Artif. Intrn. Organs 1962 8:246-251; Pateras et al. Trans. Am. Soc. Artif. Intrn. Organs 1965 11:292-295; Shimizu et al. Chemical Abstracts 1955 103:129004; Kjellstrand et al. Trans. Am. Soc. Artif. Intern. Organs 198127:24-29; and Kolff, W. J. Kidney Int. 1976 10:S211-S214).

The human gastrointestinal tract harbors a complex microbial ecosystem containing a large number and variety of bacteria. The resident bacterial population in the human gastrointestinal tract has a major impact on gastrointestinal function and thereby on human health and well being. Among these, some bacteria are opportunistic or considered to be detrimental and cause adverse conditions such as diarrhea, infections, gastroenteritis and endotoxaemia, while some bacteria species are considered as “probiotic”, in that they perform beneficial functions for the human organism (Holzapfel WH, et al. Int J Food Microbiol 1998 May 26; 41(2): 85-101).

Among the probiotic bacteria, Bifidobacteria species are the most prominent. Bifidobacteria species, when in live and viable form, stimulate the immune system and exert a competitive exclusion of pathogenic and putrefactive bacteria, reduce the amounts of ammonia and cholesterol in the blood, and promote absorption of minerals. In addition, Bifidobacteria have been suggested to exert a preventive action against colon cancer, by reducing the activity of some enzymes that convert procarcinogen substances into carcinogen substances (von Wright, et al. Eur J Gastroenterol Hepatol 1999 November; 11(11): 1195-1198).

The lactic bacteria such as Lactobacillus bulgaricus, Lactobacillus acidophilus, Lactobacillus casei, Lactobacillus plantarum and Streptococcus faecium. Streptococcus thermophilus are also probiotic. These bacteria produce antagonist effects against pathogenic microorganisms, stimulate the immune system, improve lactose digestion, perform a lypolytic activity making fats more digestible, reduce plasmatic values of cholesterol, protect the intestinal mucosa ensuring an even assimilation of the nutritive substances, produce polysaccharides that are active on some tumors, and reduce viability of some enzyme-producing microorganisms catalyzing conversion of procarcinogen substances into carcinogenic substances.

It is believed that the probiotic bacteria exert their effects in a synergistic manner to curtail and retard the growth of pathogenic/detrimental bacteria of the gut (Marteau, P R et al. Am J Clin Nutr February; 73(2 Suppl): 430S-436S; Cummings J H, et al. Am J Clin Nutr 2001 February; 73(2 Suppl): 415S-420S).

The intestinal bacteria flora can be reduced, become unbalanced or be eliminated in patients undergoing antibiotic treatment and other therapies, and in individuals suffering from inflammatory intestinal diseases, kidney disease and liver disease. In addition, it has been shown that during normal aging the Bifidobacteria population is reduced while the concentration of pathogenic and putrefactive bacteria concomitantly increases (Orrhage K., et al. Drugs Exp Clin Res 2000; 26(3): 95-111).

U.S. Pat. No. 5,733,568 teaches the use of microencapsulated Lactobacillus bacteria for treatment of antibiotic associated or other acute and chronic diarrhea as well as for skin and vaginal yeast infections. The microencapsulation is said to prevent inactivation of the bacillus and to deliver it to the intestine as well as to avoid lactose intolerance seen in said diarrheas.

U.S. Pat. No. 5,032,399 teaches the use of species of Lactobacillus acidophilus to adhere to intestinal mucosa and thereby reduce gastrointestinal side effects of antibiotic therapy that reduces beneficial bacteria population.

U.S. Pat. No. 5,531,988 teaches, in addition to beneficial bacteria, use of immunoglobulin in the composition as a dietary supplement.

U.S. Pat. No. 5,840,318 also teaches a beneficial bacterial composition that can modulate the immune system of animals.

Use of probiotics such as Lactobacillus acidophilus has been suggested to curtail the bacterial overgrowth and the accumulation of uremic toxins and carcinogenic compounds. Unabsorbable carbohydrate in the diet of uremic patients has also been shown to increase fecal nitrogen. Use of lactulose and dietary fiber has also been shown to reduce plasma urea 11 to 27% and increase fecal nitrogen excretion to 39 to 62% (Wrong, O., Nature Medicine 2-3, 1997).

One of the major deficits of these prior art approaches, however, is that they tend to address individual uremic solutes or toxins. However, proper clinical management of renal, hepatic and gastrointestinal diseases or disorders actually requires alleviation of multiple symptoms. The present invention provides a composition and method for enteric dialysis.

### SUMMARY OF THE INVENTION

An object of the present invention is to provide a composition for enteric dialysis comprising a probiotic microbe which reduces the urea concentration when ingested by a host.

The present invention further provides a method of reducing urea concentration in a host comprising administering a composition for enteric dialysis comprising a probiotic microbe which reduces the urea concentration to a host suffering from elevated uremic toxins accumulated in the blood.

### BRIEF DESCRIPTION OF THE DRAWINGS

FIG. 1 shows the survival rate of three S. thermophilus strains in simulated gastric juice at varying pH values.

FIGS. 2A through 2C show the urea hydrolysis by S. thermophilus strains at varying pH values.

FIGS. 3A and 3B show the dependency rate of urea hydrolysis on the availability of nitrogen.

FIG. 4 shows ureolysis and urea-nitrogen concentration rates for different bacterial strains FIGS. 5A through 5C show urea hydrolysis by S. thermophilus strains at concentrations of urea characteristic of uremic blood levels.

### DETAILED DESCRIPTION OF THE INVENTION

In kidney failure there is a decrease in the glomerular filtration rate and the kidneys are unable to maintain homeostasis of the blood. Homeostatic balance of water, sodium, potassium, calcium and other salts is no longer possible and nitrogenous wastes are not excreted. Retention of water causes edema and as the concentration of hydrogen ions increases, acidosis develops. Nitrogenous wastes accumulate and a condition referred to as uremia develops in the blood and tissue. Uremic toxins can be defined as solutes that: (I) are normally excreted by healthy kidneys, (ii) accumulate progressively during the development of renal failure so that their concentration increases, and (iii) inhibit various physiologic and biochemical functions; as a whole, they contribute to a complex set of clinical symptoms that comprise the Uremic Syndrome. Examples of uremic toxins include, but are not limited to, ammonia, urea, creatinine, phenols, indoles, and middle molecular weight molecules. More specifically, in uremia, the concentration of serum creatinine, blood urea nitrogen (BUN), uric acid, and guanidino compounds such as N-methyl guanidine (NMG) and guanidino succinic acid, (GSA) are significantly altered with accompanying abnormalities in acid-base equilibrium, electrolytes and water retention. In addition there are several known and unknown substances of low and middle molecular weight which have been identified as uremic toxins which also accumulate. If untreated the acidosis and uremia can cause coma and eventually death.

The introduction of renal dialysis has contributed to rapid progress in the clinical treatment of renal failure and elucidation of uremia. When a patient has mild kidney failure where the serum creatinine level is less than 400 mmol/L, the patient does not require renal replacement therapy such as dialysis or renal transplant. However, in general, when the serum creatinine level rises to 900 μmol/L, the patient needs routine dialysis or a kidney transplant to survive.

Dialysis can serve as a lifetime therapy for ESRD patients. Phosphate binders such as calcium acetate, calcium carbonate or aluminum hydroxide are generally prescribed for uremic patients receiving dialysis to reduce elevated phosphate levels. In general, however, dialysis is very expensive, inconvenient, time consuming and may occasionally produce one or more side effects. With a successful kidney transplant, a patient can live a more normal life with less long-term expense. However, there are also high costs associated with transplant surgery, the recovery period and the continuous need for anti-rejection medications. Further, there are often times a shortage of suitable donors. Accordingly there is a need for alternative strategies.

Nitrogenous wastes which accumulate in uremia flow into the gut by diffusion. The present invention provides a formulation of commensal and food grade bacteria known as probiotic bacteria, that when ingested become intestinal or gut flora and catabolize uremic toxins. Instillation of such probiotic bacteria permit reduction in frequency and even elimination of the need for dialysis. Probiotic bacteria of the present invention are living microorganisms that are naturally present in the gastrointestinal tract of humans and animals. They are beneficial bacteria that enhance the body's defenses against a number of health conditions.

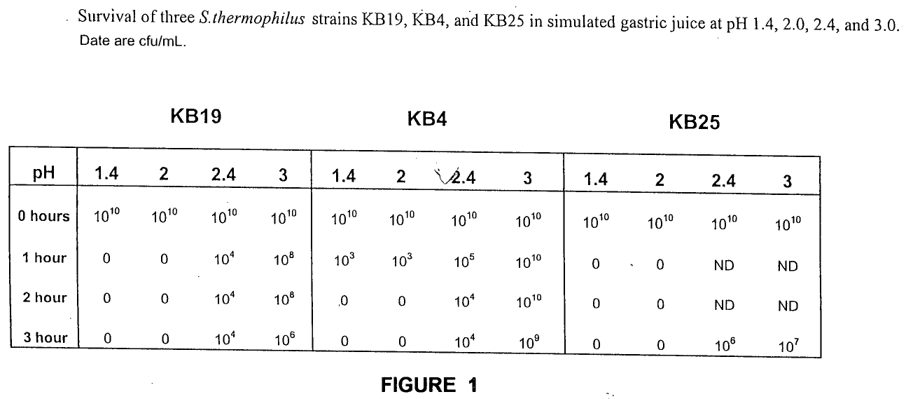

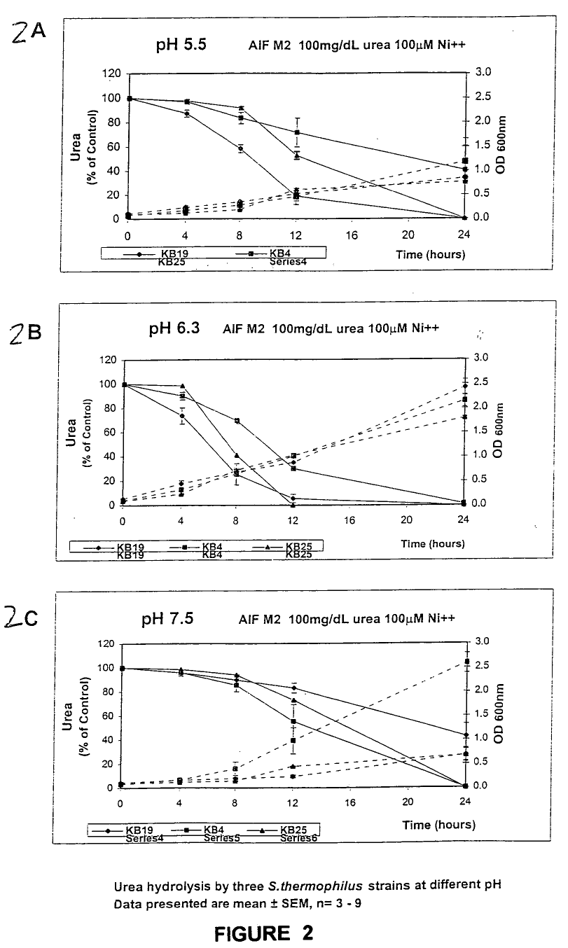

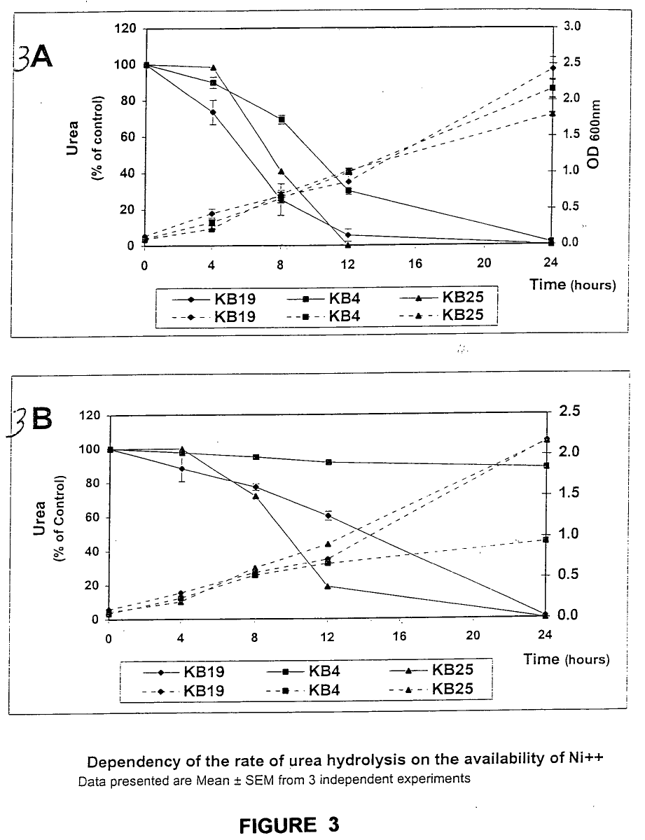

Three such probiotic bacteria, urealytic isolates of gram-positive lactic acid producing non-pathogenic cocci Streptococcus thermophilus from different sources, namely strains KB4, KB19 and KB25 were characterized in vitro by assessing their ability to catabolize urea while proliferating in the simulated gastric juice, see FIG. 1, and in simulated intestinal fluid, see FIGS. 2A through 2C. All three strains studied: proliferated in the fed state simulated artificial intestinal fluid (AIF) in the pH range from 5.5 to 7.5 characteristic of the colon environment; used urea as a sole nitrogen source, see FIGS. 3A through 3B; and catabolized urea in the presence of other nitrogen sources. Urea hydrolysis was growth- and pH-dependent. FIGS. 5A through 5C show that KB19, KB4, and KB25 respectively, are efficient in hydrolyzing urea in blood. Under all the conditions tested, the rate of urea hydrolysis was strain dependent permitting selection of the best candidate for uremic applications. One selected isolate S. thermophilus KB19, reduced urea concentrations from 300 mg/dL, to 20 mg/dL within 24 hours at pH 6.3 when inoculated at initial density of 10⁹ cfu/mL KB19 survived 3 hours in acidic pH 3.0 with only two logs loss in cfu and was able to pass through bile. In addition, this KB19 strain evidenced no resistance to commonly used antibiotics. These data indicate that a specifically selected bacterial isolate can be used as a urea-targeted component in an enteric dialysis formulation.

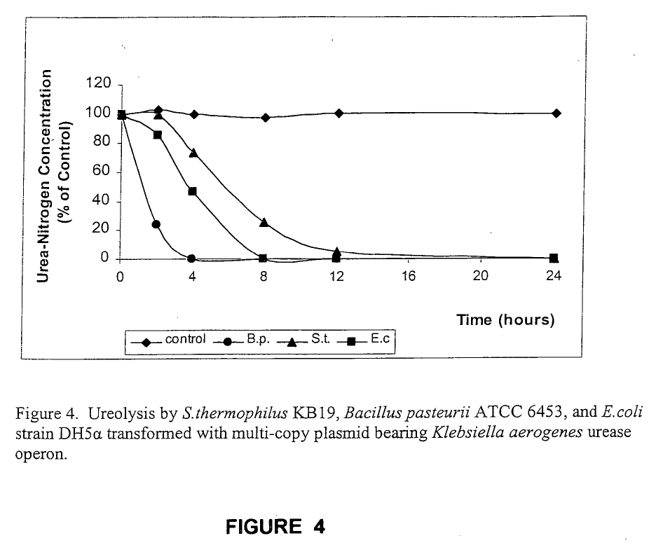

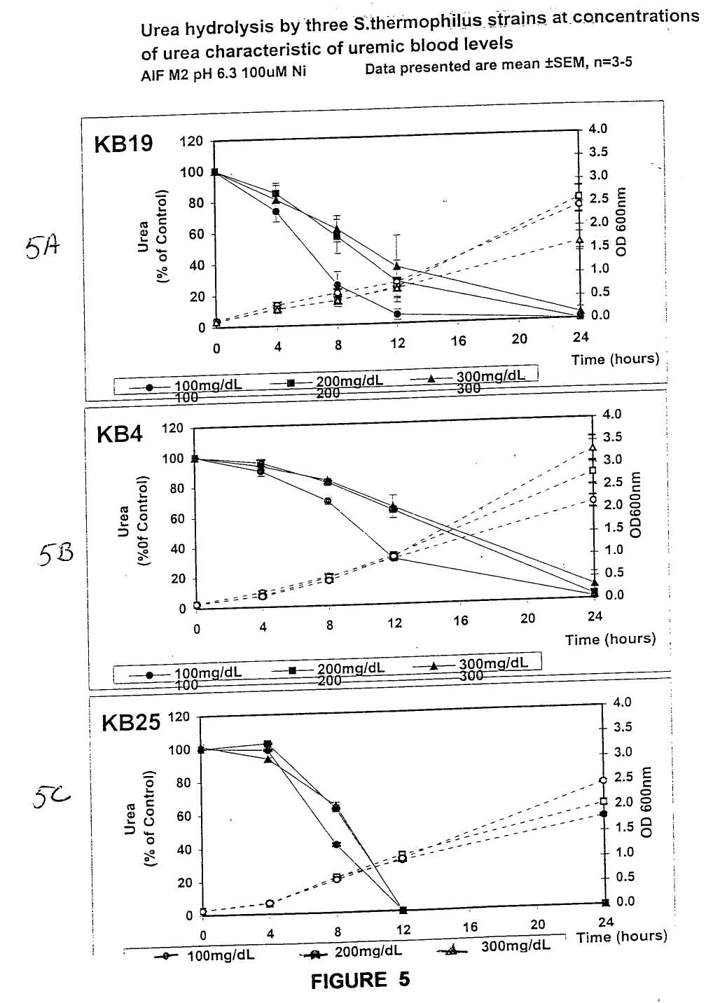

The present invention provides compositions comprising at least one strain of probiotic microbe which when ingested by the host, utilize various nitrogenous solutes. The compositions of this invention have been found to improve the patient's overall health. Microbes which have urea utilizing abilities include B. pasteurii, and genetically engineered urealytic E. coli, which have been found to be comparable to S. thermophilus KB19, see FIG. 4.

Urea hydrolysis was assessed in artificial intestinal fluid (AIF). AIF was prepared according to U.S. pharmacopocia with modifications (addition of 1% dextrose, 100 micrometers NiCl₂, 10% MRS broth and 100 mg/dL urea and, for growth of the plasmid bearing E. coli, 0.01% ampicillin). Bacterial strains were inoculated into modified AIF at the initial density of 10⁹ cfu/mL for S. thermophilus and B. pasteurii and 10⁸ cfu/mL for E. coli and incubated at 37° C., see FIG. 4. Aliquots were taken at 0, 2, 4, 8, 12 and 24 hours, urea nitrogen and optical density (OD) data was recorded.

Within 24 hours all strains removed 100% of urea from the system. Growth was recorded as proportional to OD 600 nm reading on a relative scale shown below. Both genetically engineered E. coli and soil borne bacterium B. pasteurii exhibit high rates of urea hydrolysis but neither are Generally Recognized As Safe (GRAS) by the FDA and therefore cannot be used for human consumption. However, S. thermophilus KB19 has GRAS status and based on the data presented can remove urea almost with comparable efficiency in vitro.

The present invention relates to ingestible compositions comprising one or a mixture of probiotics. The compositions can further comprise sorbents with specific adsorption affinities for uremic toxins such as creatinine, uric acid, phenols, indoles, middle molecular weight molecules and inorganic phosphate along with a water sorbent, for use in the alleviation of uremia. In another embodiment, the composition may comprise one or more of the following: a probiotic bacteria, a prebiotic such as inulin, a fructan oligosaccharide, lactulose and other vegetable fibers, an ammoniaphilic urea degrading microorganism with high alkaline pH stability and high urease activity, adsorbents such as locust bean gum with a specific adsorption affinity for creatinine and urea, activated charcoal with a specific adsorption affinity for creatinine, guanidines, phenol, indican and middle molecular weight undefined components, or water absorbents such as psillium fiber, guar gum and locust bean gum.

The bacterial source for the probiotic bacteria may be capable or metabolizing urea and ammonia, preferably to amino acids which can be used by the bacteria or the patient.

In some embodiments, the probiotic may function to restore normal balance between beneficial bacteria and detrimental bacteria, to remove excess urea-waste product of normal protein metabolism thereby reducing the burden on ailing kidney, and to remove ammonia to avert mental retardation and related conditions, as well as to act as the ammoniaphilic urea degrading microorganism. Thus, the probiotic and the ammoniaphilic urea degrading organism comprise the same species of bacteria.

Compositions comprising these probiotic bacteria may be enteric coated and/or microencapsulated. Enteric coating of some or a part of the allows the probiotic bacterial source to be delivered at the ileal and colonic regions of the bowel where maximal resorption of uremic solutes and other molecules are found to occur. This is preferably achieved via an enteric coating material that disintegrates and dissolves at a pH of 7.5 or higher. Examples of enteric coatings with these characteristics include, but are not limited to, Zein, polyglycolactic acid, polylactic acid, polylactide-coglycolide and similar coating materials. Enteric coatings also enable delivery of the sorbents to their site of action in relatively native form without binding of various digestive materials to the sorbents prior to reaching the target region.

In a preferred embodiment, oral delivery of the compositions is accomplished via an emulsion or paste mixed with an easy to eat food. The oral delivery of the compositions may be via ready to eat food or other nutritional product. The delivery of the compositions of the present invention may be via pharmaceutical compositions of liquid, capsule, pill or other suitable forms. The probiotic bacteria can be administered along with a mixture of sorbents in the emulsion or paste or separately in an ingestible capsule.

What is claimed is:

1. A composition for enteric dialysis comprising a probiotic bacteria which reduces the urea concentration when ingested by a host.

2. The composition of claim 1 wherein the probiotic bacteria is Streptococcus thermophilus strain KB4.

3. The composition of claim 1 wherein the probiotic bacteria is Streptococcus thermophilus strain KB19.

4. The composition of claim 1 wherein the probiotic bacteria is Streptococcus thermophilus strain KB25.

5. A method of reducing urea concentration in a host comprising administering the composition of claim 1 to a host suffering from elevated uremic toxin accumulated in the blood.
